(19) United States
(12) Patent Application Publication
(10) Pub. No.: US 2014/0113244 A1
(43) Pub. Date: Apr. 24, 2014

(54) TOOTH WHITENING COMPOSITION CONTAINING COLLOIDAL PLATINUM

(71) Applicant: REGENETISS, INC., Nagano (JP)

(72) Inventors: Toshikazu Shiba, Nagano (JP); Yumi Kawazoe, Nagano (JP); Shinichi Katou, Nagano (JP)

(73) Assignee: REGENETISS, INC., Nagano (JP)

(21) Appl. No.: 14/123,907

(22) PCT Filed: Sep. 26, 2012
(86) PCT No.: PCT/JP2012/074727

§ 371 Date: Dec. 4, 2013

(30) Foreign Application Priority Data
Sep. 30, 2011 (JP) 2011-216188

(51) Int. Cl. A61K 8/22; A61K 8/19; A61Q 11/00; A61N 5/06; A61C 19/06

(52) U.S. Cl. CPC A61K 8/22; A61N 5/062; A61N 5/0603; A61C 19/066; A61Q 11/00; A61K 8/19
USPC 433/29; 433/216; 424/53

(57) ABSTRACT

A tooth whitening composition with a high level of whitening effectiveness over the entire tooth whitening composition application includes colloidal platinum as a platinum catalyst in a peroxide-containing oral composition. Whitening effectiveness can be enhanced by application of a tooth whitening composition containing colloidal platinum to the teeth surface followed by photo-irradiation of the application site.

### TECHNICAL FIELD

The present invention relates to a tooth whitening composition containing colloidal platinum.

### BACKGROUND ART

A surface of the teeth is colored with a coloring causative agent such as coffee, tea, and tobacco tar. It is a goal of dentistry for many years to remove the stain from the teeth surface and to keep the teeth white, and thus many products for whitening of the teeth surface have been developed. In addition, treatment for whitening of the teeth surface by a dentist has also performed.

Conventionally, in order to whiten the teeth surface, an oral composition containing peroxide has been used. It has been known that peroxide itself has a whitening effect by its decomposition. The decomposition of the peroxide is accelerated by a metal catalyst such as titanium dioxide, as a result, the peroxide improves a whitening effect. As described above, an oral composition containing both peroxide and a metal catalyst has been used for tooth whitening.

In Patent Literature 1, there is a disclosure of platinum-supported titanium oxide, and a tooth whitening composition containing peroxide. In this Literature, it is described that the platinum-supported titanium oxide has high decomposition efficiency of peroxide. Therefore, by using the tooth whitening composition disclosed in Patent Literature 1, the desired teeth whitening effect can be obtained in a shorter time than before.

In Patent Literature 2, there is a disclosure of a composition for teeth whitening composition containing a catalyst activator and a bleaching agent such as peroxide. In this Literature, it is described that platinum or a platinum salt can be used as a catalyst activator.

### CITATION LIST

### Patent Literature

Patent Literature 1: Japanese Patent Application Laid-Open No. 2008-189548
Patent Literature 2: Japanese Patent Application National Publication No. 2002-514225

### SUMMARY OF INVENTION

### Technical Problem

However, the whitening effect of the oral composition containing a catalyst such as titanium dioxide, platinum, or a platinum salt, and peroxide was not sufficient. In addition, platinum only dissolves in a mixture solution of concentrated nitric acid and concentrated hydrochloric acid, and further, as to the platinum salt, there is also a platinum salt that does not dissolve in an aqueous solution depending on the kind of the salt. Therefore, when the platinum or the platinum salt was mixed into an oral composition as it is, there was the platinum or the platinum salt remained precipitated powders without being thoroughly dissolved in the oral composition.

Further, a compound in which platinum is supported on titanium oxide was hardly dissolved in water either, and thus there was the platinum remained precipitated powders without being thoroughly dissolved in the oral composition.

When platinum, a platinum salt, or a compound in which platinum is supported was precipitated in an oral composition, the platinum catalyst is not uniformly dispersed, and thus is not uniformly applied on teeth surface. Therefore, when the oral composition is applied on teeth surface, there was a problem that a sufficient whitening effect cannot be obtained over the entire applied area. Therefore, in recent years, an oral composition in which high whitening effect can be obtained over the entire applied area is required.

### Solution to Problem

The present inventors obtained the findings that a tooth whitening composition having a high whitening effect over the entire applied area can be obtained by the inclusion of colloidal platinum as a platinum catalyst in an oral composition containing peroxide. Further, the present inventors also obtained the findings that the whitening effect can be enhanced by the application of a tooth whitening composition containing colloidal platinum on teeth surface and then by the irradiation of the applied area with light.

The present invention relates to a tooth whitening composition. The tooth whitening composition according to the present invention contains 0.01% by weight or more to 5% by weight or less of colloidal platinum, and 1% by weight or more to 30% by weight or less of peroxide. A tooth whitening composition having high whitening effect can be obtained by the addition of colloidal platinum and peroxide to a tooth whitening composition.

The tooth whitening composition according to the present invention may further contain 0.1% by weight or more to 30% by weight or less of phosphate polymer, a salt of phosphate polymer, or a solvate of phosphate polymer. A tooth whitening composition having higher whitening effect can be obtained by the addition of phosphate polymer to a tooth whitening composition.

In a preferred embodiment of the present invention, the phosphate polymer, the salt of phosphate polymer, or the solvate of phosphate polymer in a tooth whitening composition is ultraphosphate, a salt of ultraphosphate, or a solvate of ultraphosphate, respectively. With the addition of ultraphosphate to a tooth whitening composition, a tooth whitening composition having higher whitening effect can be obtained.

In a preferred embodiment of the present invention, the peroxide is hydrogen peroxide, carbamide peroxide, or calcium peroxide. With the use of these peroxides, a tooth whitening composition having safety and higher whitening effect can be obtained.

In a preferred embodiment of the present invention, the pH of a tooth whitening composition is 6 or more to 8 or less. The pH of a tooth whitening composition is neutral, therefore, the tooth whitening composition according to the present invention can perform the whitening of the teeth without giving a stimulus to other organs such as the gums in the oral cavity.

In a preferred embodiment of the present invention, the tooth whitening composition is used by the application of the tooth whitening composition on teeth surface, and then by the irradiation of the applied area with light having a wavelength of 420 nm or more to 750 nm or less. By the irradiation with light, the whitening of the teeth surface can be performed more effectively.

In a preferred embodiment of the present invention, the tooth whitening composition is used by the application of the tooth whitening composition on teeth surface, and then by the irradiation of the applied area with first light having a wavelength of 420 nm or more to 490 nm or less and second light having a wavelength of 620 nm or more to 640 nm or less. By the irradiation with lights having two different wavelengths, whitening of the teeth surface can be performed more effectively.

In a preferred embodiment of the present invention, the tooth whitening composition is used by the application of the tooth whitening composition on the teeth surface, and then by the irradiation with light having a wavelength of 800 nm or more to 1500 nm or less. By the irradiation with light having long wavelengths, the high whitening effect can be obtained in a short time.

### Advantageous Effects of Invention

According to the present invention, with the cleaning of teeth surface with a tooth whitening composition containing colloidal platinum and peroxide, high whitening effect can be obtained over the entire applied area of the tooth whitening composition.

### BRIEF DESCRIPTION OF THE DRAWINGS

FIG. 1 shows the changes of color residual ratio when various kinds of catalysts are used, in which irradiation with light has not been performed.

FIG. 2 shows the changes of color residual ratio when various kinds of catalysts are used, in which irradiation with light has been performed.

### DESCRIPTION OF EMBODIMENT

The tooth whitening composition according to the present invention is a composition that removes stain from the teeth and whitens the teeth surface. That is, the tooth whitening composition is a composition that is applied mainly on teeth surface in the oral cavity, removes stain from the teeth, and whitens the teeth surface. The tooth whitening composition according to the present invention can be used for the cleaning of a full denture and a denture.

The tooth whitening composition according to the present invention may be contained in toothpaste or mouthwash. Further, the tooth whitening composition according to the present invention may be in liquid or gel form. The tooth whitening composition according to the present invention may be used in whitening performed by a dentist.

It has been known that platinum has high active oxygen removal capacity. It has been considered that platinum is not absorbed into the body through the skin or the intestine. Therefore, platinum is a metal that is allowed to be ingested orally in order to remove the active oxygen on the surface of the skin or in the intestine.

Platinum does not dissolve in an aqueous solution, and dissolves only in a mixture solution of concentrated nitric acid and concentrated hydrochloric acid. Further, it has been known that the platinum salt is also slightly soluble in an aqueous solution. Therefore, platinum and a platinum salt are precipitated in an aqueous solution, and are not uniformly dispersed in an aqueous solution.

Colloidal platinum is made of the minute particles of platinum that are processed to the size of from several nanometers to several tens of nanometers in diameter. The colloidal platinum of the present invention, for example, has an average particle size of from 1 nm to 5 nm, and the particle sizes of 90% or more of the colloidal platinum are in the range of from 0.1 nm to 10 nm. The colloidal platinum may have an average particle size of from 1 nm to 50 nm, preferably an average particle size of from 1 nm to 10 nm, more preferably an average particle size of from 1 nm to 5 nm, and furthermore preferably an average particle size of from 1.5 nm to 2.5 nm. Further, the particle sizes of 90% or more of the colloidal platinum are preferably in the range of from 1 nm to 10 nm, more preferably in the range of from 1 nm to 5 nm, and furthermore preferably in the range of from 1 nm to 3 nm. With the use of the colloidal platinum having a narrow particle size distribution, the colloidal platinum can be uniformly dispersed in a tooth whitening composition. Further, for the measurement of the particle sizes of colloidal platinum, a known dynamic light scattering-type particle size distribution measurement method can be used. Further, the particle sizes of colloidal platinum may be measured by a laser diffraction measurement method, an X-ray diffraction method, or the like.

The colloidal platinum can also be contained in a colloidal platinum solution. The colloidal platinum solution is a solution that contains colloidal platinum and sodium polyacrylate. The colloidal platinum solution is a uniform dispersion of colloidal platinum. In the colloidal platinum solution, a polyacrylic acid salt is coordinated to platinum, and becomes a colloidal protective agent that improves the solvent affinity of the platinum. Therefore, the colloidal platinum can be more uniformly dispersed without being aggregated.

R-value in the colloidal platinum solution is preferably 80 to 180, more preferably 90 to 170, and furthermore preferably 100 to 150. The R-value shows a ratio of the mole number of the colloidal protective agent and the mole number of the platinum. When the R-value is 80 to 180, even in an ionic solution added with cations and the like, the colloidal platinum can maintain the dispersion state.

In the present invention, colloidal platinum is contained in a tooth whitening composition in an amount of 0.01% by weight or more to 5% by weight or less, preferably 0.02% by weight or more to 2%, by weight or less, and more preferably 0.05% by weight or more to 1% by weight or less. In the present invention, the platinum is made into colloidal form and thus has a large surface area, therefore, even the content of the colloidal platinum is small, the whitening effect can be exerted.

Further, the colloidal platinum and the colloidal platinum solution can be produced by using a known method.

The colloidal platinum itself removes the active oxygen. On the contrary, colloidal platinum can also be used as a catalyst that enhances the whitening effect with peroxide in the present invention. In a tooth whitening composition containing colloidal platinum and peroxide, the colloidal platinum is uniformly dispersed in the tooth whitening composition, therefore, the high whitening effect is exerted. In the present invention, a tooth whitening composition containing colloidal platinum and peroxide is applied on the teeth surface, and left for 15 minutes to 30 minutes, and then the tooth whitening composition is removed, as a result, the stain on the teeth surface is removed. In addition, after the application of a tooth whitening composition containing colloidal platinum and peroxide on teeth surface, by the irradiation of the applied area with light, the higher whitening effect can be obtained.

Further, in the present invention, a tooth whitening composition containing colloidal platinum and peroxide is applied in the oral cavity, functions to whiten the surface of the teeth, whereas colloidal platinum itself removes the active oxygen in the oral cavity. In addition, a tooth whitening composition containing colloidal platinum and peroxide also functions effectively for the treatment and prevention for gingivitis, periodontal disease, and the like.

The peroxide is a compound containing peroxide ions (O₂²⁻), and is a strong oxidant. Examples of the peroxides used in the present invention include hydrogen peroxide, carbamide peroxide, and calcium peroxide. The concentration of peroxide may be 1% by weight or more to 20% by weight or less, preferably 3.5% by weight or more to 15% by weight or less, more preferably 3.5% by weight or more to 10% by weight or less, and furthermore preferably 5% by weight or more to 10% by weight or less. With the addition of the peroxide to the tooth whitening composition in the above-described range, the whitening effect of the tooth whitening composition of the present invention can be enhanced.

The tooth whitening composition according to the present invention may contain phosphate polymer. Examples of the phosphate polymer include polyphosphate, a salt of polyphosphate, or a solvate of polyphosphate. The polyphosphate, salt of polyphosphate, or solvate of polyphosphate to be used may be one kind, or may be a mixture of multiple kinds. In the multiple kinds of polyphosphate, a salt of the polyphosphate, or a solvate of the polyphosphate, the polyphosphate, a salt of the polyphosphate, or a solvate of the polyphosphate which has different chain length or different molecular structure, and the polyphosphate having different metal ion are contained.

The salt of polyphosphate means a salt of polyphosphate, particularly a salt of pharmaceutically acceptable polyphosphate. As a salt of polyphosphate, an alkali metal salt is preferred, and a sodium salt is more preferred. In the present specification, a salt of polyphosphate may contain not only anhydrite but also a hydrate salt. These salts, for example, are ionized in a solution and function in the same way as in polyphosphate.

The solvate of polyphosphate means a solvate of polyphosphate, or a solvate of a salt of polyphosphate. As the solvate, a hydrate can be mentioned. Further, there may be a case that the polyphosphate used in the present invention absorbs moisture in the tooth whitening composition, and thus becomes a hydrate by being attached with the adsorbed water. Even in the case of forming such a solvate, the solvate is included in the solvates of polyphosphate. The above-described solvates are ionized in a solution and function in the same way as in polyphosphate.

In the present invention, the phosphate polymer contains preferably 0.1% by weight or more to 30% by weight or less, more preferably 0.5% by weight or more to 20% by weight or less, and furthermore preferably 1% by weight or more to 10% by weight or less, on the basis of the total weight of the tooth whitening composition. The polyphosphate may be a salt of the polyphosphate, or a solvate of the polyphosphate, the content percentage of polyphosphate for the tooth whitening composition is a content percentage of the total mass of these three kinds of polyphosphate.

Among the polyphosphates, ultraphosphate is preferable. The ultraphosphate is polyphosphate that is a phosphoric acid polymer having a highly branched (mesh) structure. A preferable example of the ultraphosphate is ultraphosphate having an average chain length of which is 5 or more to 25 or less phosphate residues. Further, the average chain length of ultraphosphate may be 7 or more to 20 or less, or may be 7 or more to 15 or less. In particular, when the average chain length of ultraphosphate is 7 or more to 12 or less, a higher whitening effect can be obtained due to the synergistic effect of peroxide.

The pH of the tooth whitening composition according to the present invention may be 6 or more to 8 or less. The tooth whitening composition according to the present invention exerts the effects even in the neutral condition of the pH. Further, the pH of the tooth whitening composition is neutral, therefore, the tooth whitening composition according to the present invention can be used for performing the whitening of the teeth without giving a stimulus to other organs such as the gums in the oral cavity.

The tooth whitening composition according to the present invention is applied on teeth surface, and stood still for 3 minutes to 60 minutes, preferably for 5 minutes to 40 minutes, and more preferably 10 minutes to 30 minutes. After that, the tooth whitening composition is removed from the teeth surface. With the application of a tooth whitening composition containing colloidal platinum and peroxide on teeth surface, the peroxide is decomposed on the teeth surface, and thus the whitening effect can be exerted. Here, the colloidal platinum functions as a catalyst for the decomposition of the peroxide. In the tooth whitening composition according to the present invention, the colloidal platinum is uniformly dispersed, therefore, the colloidal platinum is uniformly applied on teeth surface, and thus uniform whitening effect can be exerted.

The tooth whitening composition according to the present invention may be used by the application of the tooth whitening composition on teeth surface, and then by the irradiation of the applied area with light having a wavelength of 420 nm or more to 750 nm or less. Examples of the intensity of the light to be emitted include 1 W or more to 50 W or less, and may be 3 W or more to 20 W or less, or 5 W or more to 10 W or less. The time of the irradiation with light is 1 minute to 30 minutes, preferably 3 minutes to 20 minutes, and more preferably 5 minutes to 15 minutes. By the irradiation with light, colloidal platinum is easily excited, and works more effectively as a catalyst. In this case, teeth surface can be whitened more effectively, and thus the time required for the whitening can be reduced.

The tooth whitening composition according to the present invention may be used by the application of the tooth whitening composition on teeth surface, and then by the irradiation of the applied area with first light having a wavelength of 420 nm or more to 490 nm or less and second light having a wavelength of 620 ran or more to 640 nm or less. Examples of each intensity of the first light and second light to be emitted include 1 W or more to 50 W or less, and may be 3 W or more to 20 W or less, or 5 W or more to 10 W or less. By the irradiation with lights having two different wavelengths, the colloidal platinum works more effectively as a catalyst, and thus effective whitening treatment can be performed.

By the irradiation with two types of lights, not only the whitening effect is enhanced but also the teeth surface can be whitened in a shorter time, and thus the load on the user at the time of application is reduced.

The tooth whitening composition according to the present invention may be used by the application of the tooth whitening composition on teeth surface, and then by the irradiation of the applied area with an infrared ray. Examples of the wavelength of the infrared ray include 750 nm or more to 2000 nm or less, and may be 800 nm or more to 1500 nm or less, 800 nm or more to 1300 inn or less, or 850 nm or more to 1050 nm or less. Examples of the intensity of the infrared ray to be emitted include 1 W or more to 50 W or less, and may be 3 W or more to 20 W or less, or 5 W or more to 10 W or less.

The tooth whitening composition is applied on teeth surface, and then the irradiation of the applied area with laser light having long wavelengths is performed, thus the high whitening effect can be obtained in a short time.

The present invention can also be a whitening method of the teeth in which after the application of a tooth whitening composition on the teeth, the applied area is stood still for 1 minute to 60 minutes, preferably for 5 minutes to 40 minutes, and more preferably 10 minutes to 30 minutes, and then the tooth whitening composition is removed. Further, the whitening effect can be enhanced by the irradiation with light having a wavelength in each wavelength range described above.

The tooth whitening composition according to the present invention is generally stayed in the oral cavity for a certain period of time to clean the inside of the oral cavity, to prevent the oral cavity from drying, and to give a refreshing feel and the like, or has a preventive or treatment effect for disease in the oral cavity. Examples of a mouth cleaning agent include toothpaste, abrasive, mouthwash, spray-type oral cleaning agent, gargle, and oral rinse. The colloidal platinum according to the present invention is a nanoscale, therefore, is easily and uniformly dispersed into a solution. Therefore, the tooth whitening composition can also be a composition in liquid form with low viscosity.

In the tooth whitening composition, as needed, an active ingredient such as a thickening agent, a surfactant, a sweetener, a preservative, vitamins, and minerals; and an ingredient such as a coloring agent, a flavor, and an algefacient can be mixed. A tooth whitening composition containing colloidal platinum and peroxide, and a tooth whitening composition containing various kinds of ingredients can be mixed and produced in accordance with a usual method of producing toothpaste or mouthwash.

### Example 1

### Preparation of Ivory Disc

The surface of a commercially available ivory disc (circular shape, around 1.5 cm in diameter, and around 3 mm in thickness) was polished so as to smooth the surface of the disc by using 60 grit sandpaper (coarse). The whiteness on the polished surface of ivory disc was measured by using Shade Up Navi (manufactured by SHOFU INC.), and the shade (brightness) on the polished surface was confirmed to be “1”, and then the ivory disc was used for the test. The values of brightness shown by Shade Up Navi are 1 to 9, and displayed as a numerical value in increments of 0.5. Shade 1 is the highest brightness, and the numerical value becomes larger, the brightness is lowered.

Preparation of Various Solutions Used for Coloring Operation

Various solutions used for coloring operation were prepared as follows. At first, IXOS SCP5000 (manufactured by Nitta Gelatin Inc.) was dissolved in a purified water so that the content is 1% by weight, and thus the protein solution was prepared. Next, Sunphenone BG (manufactured by Taiyo Kagaku Co., Ltd.) containing polyphenol derived from tea leaves was dissolved in a purified water so that the content is 20% by weight, and thus the coloring liquid of the teeth was prepared. Further, each reagent was dissolved in a purified water so that 20 mM HEPES-KOH (pH 7.0), 1.5 mM CaCl₂, and 0.9 mM KH₂PO₄ can be contained, and thus the mixture was used as an artificial saliva.

Preparation of Colored Ivory Disc as a Colored Tooth Model

At first, the polished ivory disc was immersed in the prepared protein solution, and slowly stirred at 30° C. for 15 minutes. After that, the disc was taken out from the solution, and then immersed in a coloring liquid, and slowly stirred at 30° C. for 15 minutes. Further, the disc was taken out from the liquid, and then immersed in an artificial saliva, and slowly stirred at 30° C. for 15 minutes. After the operation of immersing in a protein solution, a coloring liquid, and an artificial saliva was repeated three times in total, the ivory disc was washed thoroughly with a purified water, and dried in air, and then the brightness of each disc was measured by using Shade Up Navi. In the case that the brightness of the disc after the coloring operation was less than 6, the coloring is considered to be insufficient, and a series of coloring operation was performed again, the brightness was confirmed to be 6 or more, and then the ivory disc was used for the test.

Preparation of Whitening Composition

As a catalyst that improves the radical generation from hydrogen peroxide, a whitening composition containing 1% by weight of colloidal platinum (manufactured by Apt Co., Ltd.) and 15% by weight of hydrogen peroxide was prepared. The whitening composition further contains 100 mM phosphate buffer solution, and was adjusted to be pH 7.0 by the addition of sodium hydroxide.

### Comparative Example 1

As a catalyst that improves the radical generation from hydrogen peroxide, a tooth whitening composition containing 1% by weight of potassium chloroplatinate (manufactured by Wako Pure Chemical Industries, Ltd.) and 15% by weight of hydrogen peroxide was prepared. The whitening composition further contains 100 mM phosphate buffer solution, and was adjusted to be pH 7.0 by the addition of sodium hydroxide.

### Comparative Example 2

As a catalyst that improves the radical generation from hydrogen peroxide, a tooth whitening composition containing 1% by weight of titanium dioxide (anatase type, manufactured by Wako Pure Chemical Industries, Ltd.) and 15% by weight of hydrogen peroxide was prepared. The whitening composition further contains 100 mM phosphate buffer solution, and was adjusted to be pH 7.0 by the addition of sodium hydroxide.

### Comparative Example 3

A tooth whitening composition containing 15% by weight of hydrogen peroxide and not containing a catalyst that improves the radical generation from hydrogen peroxide was prepared. The whitening composition further contains 100 mM phosphate buffer solution, and was adjusted to be pH 7.0 by the addition of sodium hydroxide.

### Test Example 1

### Whitening Effect Evaluation Test (without Light Irradiation)

The colored ivory disc was immersed in 1 mL of a solution of whitening composition that had been prepared in Example 1 and Comparative Examples 1 to 3, and thus the whitening treatment was performed. Each whitening treatment was performed at 30° C. for 15 minutes, and at most for 30 minutes, the whitening treatment was performed. The solution containing hydrogen peroxide and various kinds of catalysts was replaced with a new one every 15 minutes. The ivory discs were taken out from the solution every 15 minutes, washed with a purified water, then dried in air, and subsequently the brightness of each disc was measured by using Shade Up Navi. As a result, the value of the brightness on the surface of each ivory disc after the whitening treatment was obtained.

In Table 1, the results of the whitening effect evaluation test performed in Test Example 1 were summarized. The value of the brightness of colored ivory disc was compared with the value of the brightness of the ivory disc after the whitening treatment, and the coloring removal rate was calculated. The values in Table 1 show around how many times the coloring removal rate of the whitening composition containing the colloidal platinum in Example 1 is increased as compared with the coloring removal rate of the whitening composition in Comparative Examples 1 to 3.

TABLE 1

| Treatment group | Ratio of coloring removal rate (times) |
|---|---|
| [Comparative Example 1] | 1.42 |
| 15% hydrogen peroxide + | |
| 1% chloroplatinic acid | |
| (without light irradiation) | |
| [Comparative Example 2] | 4.31 |
| 15% hydrogen peroxide + | |
| 1% titanium dioxide | |
| (without light irradiation) | |
| [Comparative Example 3] | 2.58 |
| only 15% hydrogen peroxide | |
| (without light irradiation) | |

As shown in Table 1, in the case without light irradiation, the removal rates of coloring in 30 minutes of the treatment were compared with each other. In the case of using a nanocolloidal platinum catalyst, there was a whitening effect improving ability 1.42 times, 4.31 times, and 2.58 times higher as compared with those in the case using potassium chloroplatinate and titanium dioxide that were not nanocolloidal, and the case not using a catalyst, respectively. As described above, it was found that by using a nanocolloidal platinum catalyst, the removal efficiency of the coloring adhered to the ivory surface is a significantly higher as compared with those of other catalysts.

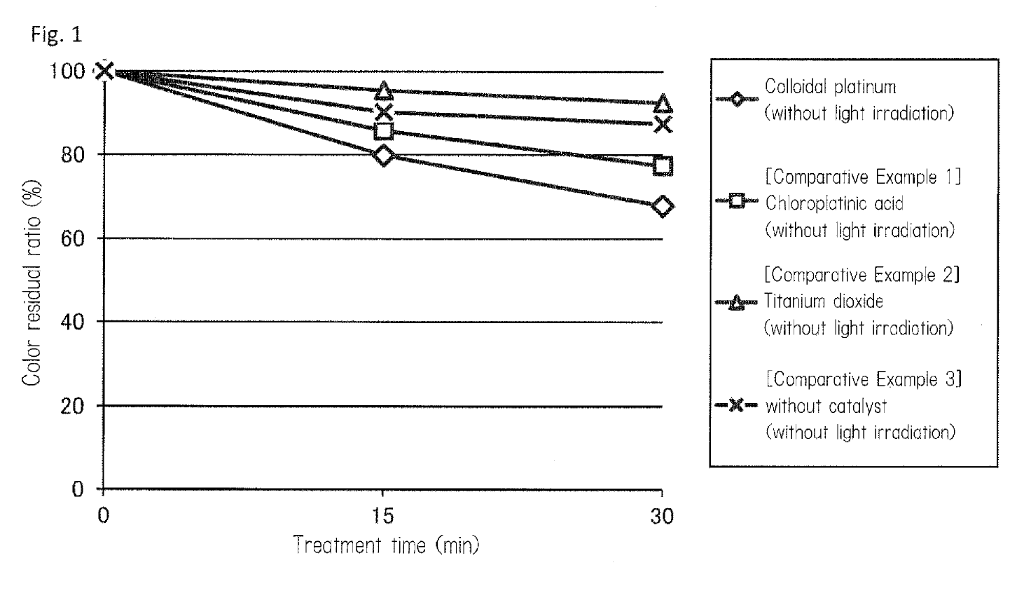

FIG. 1 shows color residual ratio after the whitening treatment in the case without light irradiation. As shown in FIG. 1, in the cases of performing the whitening treatments with a whitening composition not containing a catalyst, and with a whitening composition containing a titanium dioxide catalyst, even in 30 minutes of the treatment, the color residual ratio was 80% or more. Further, in the case of performing the whitening treatment with a whitening composition containing potassium chloroplatinate, in 30 minutes of the treatment, the color residual ratio was 77%. On the other hand, in the case of performing the whitening treatment with a whitening composition containing colloidal platinum, the color residual ratio was 68%. As described above, it was found that in the case of performing the whitening treatment with a whitening composition containing colloidal platinum, significantly higher whitening effect can be obtained as compared with those in the case of using other catalysts or of not using a catalyst.

### Test Example 2

### Whitening Effect Evaluation Test (with Light Irradiation)

In the same way as in Test Example 1, the colored ivory disc was immersed in 1 mL of a solution of whitening composition that had been prepared in Example 1 and Comparative Examples 1 to 3, and thus the whitening treatment was performed. At this time, the ivory disc was irradiated with LED light. As a light irradiator, Whitening Tooth System DualColor MD669 (manufactured by Hongda Dental, Hong Kong) was used. The irradiation was performed with the simultaneous lighting of LED lights of blue (a wavelength of 430 to 490 nm) and red (a wavelength of 620 to 640 nm). The LED light was set so that the output can be 100%. Further, the LED light was used by being fixed so that the distance of the light can be around 2 cm from the surface of the treatment liquid. The ivory discs were taken out from the solution every 15 minutes, washed with a purified water, then dried in air, and subsequently the brightness of each disc was measured by using Shade Up Navi. As a result, the value of the brightness on the surface of each ivory disc after the whitening treatment was obtained.

In Table 2, the results of the whitening effect evaluation test performed in Test Example 2 were summarized. The value of the brightness of colored ivory disc was compared with the value of the brightness of the ivory disc after the whitening treatment, and the coloring removal rate was calculated. The values in Table 2 show around how many times the coloring removal rate of the whitening composition containing the colloidal platinum in Example 1 is increased as compared with the coloring removal rates of the whitening compositions in Comparative Examples 1 to 3.

TABLE 2

| Treatment group | Ratio of coloring removal rate (times) |
|---|---|
| [Comparative Example 1] | 1.70 |
| 15% hydrogen peroxide + | |
| 1% chloroplatinic acid | |
| (with light irradiation) | |
| [Comparative Example 2] | 1.73 |
| 15% hydrogen peroxide + | |
| 1% titanium dioxide | |
| (with light irradiation) | |
| [Comparative Example 3] | 1.28 |
| only 15% hydrogen peroxide | |
| (with light irradiation) | |

As shown in Table 2, in the case with light irradiation, when removal rates of the coloring in 30 minutes of the treatment were compared, in the case of using a nanocolloidal platinum catalyst, there was a whitening effect improving ability 1.70 times, 1.73 times, and 1.28 times higher as compared with those in the cases of using potassium chloroplatinate and titanium dioxide that were not nanocolloidal, and of not using a catalyst, respectively. As described above, it was found that by using a nanocolloidal platinum catalyst, the removal efficiency of the coloring adhered to the ivory surface is a significantly higher as compared with those of other catalysts.

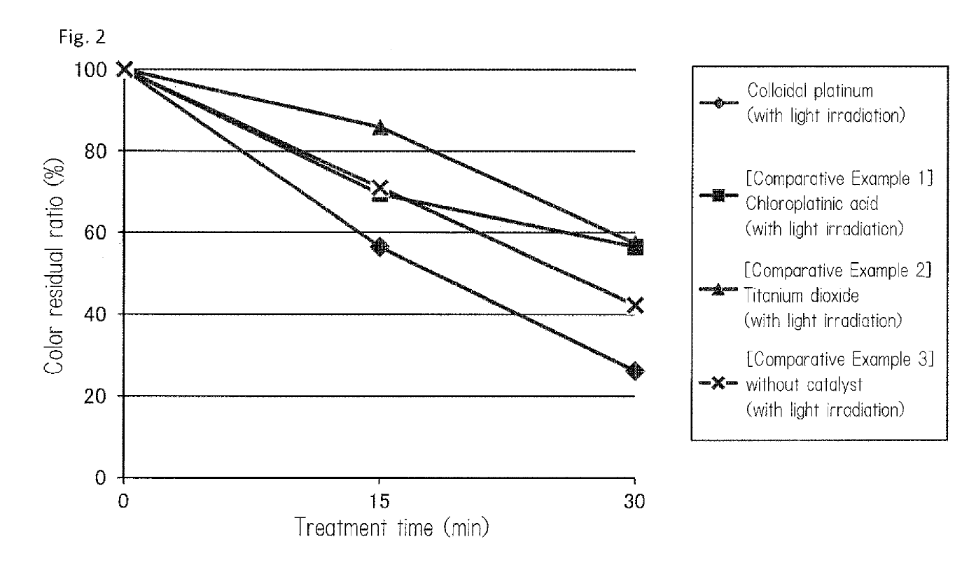

FIG. 2 shows color residual ratio after the whitening treatment in the case with light irradiation. As shown in FIG. 2, in the case of performing the whitening treatments with whitening compositions containing a titanium dioxide catalyst and a chloroplatinic acid catalyst, with light irradiation, even in 30 minutes of the treatment, the color residual ratio was 50% or more. Further, in the case of performing the whitening treatment with a whitening composition not containing a catalyst, with light irradiation, in 30 minutes of the treatment, the color residual ratio was 42%. On the other hand, in the case of performing the whitening treatment with a whitening composition containing colloidal platinum, the color residual ratio was 26%. As described above, it was found that in the cases of performing the whitening treatment with a whitening composition containing colloidal platinum, significantly higher whitening effect can be obtained as compared with those in the case of using other catalysts or of not using a catalyst.

In addition, it was found that also in the case of performing the whitening treatment with a whitening composition containing colloidal platinum, significantly higher whitening effect can be obtained by performing the irradiation with light.

### Example 2

### Preparation of Tea-Stained Dry Hydroxyapatite

At first, each of a protein solution (1% IXOS SCP5000, manufactured by Nitta Gelatin Inc.), a stain solution, and an artificial saliva [20 mM HEPES KOH (pH 7.0), 1.5 mM CaCl₂, 0.9 mM KH₂PO₄] was prepared.

The stain solution was prepared as follows. Five-grams of instant coffee, 2 tea bags, and 2 green tea bags were put into 110 mL of water, and the mixture was heated for 2 minutes in a microwave oven. After that, the resultant was allowed to be radiated for 3 hours to overnight while shaking at room temperature. After that, the resultant solution was filtered through a 200 mesh filter paper.

The tea-stained dry hydroxyapatite was prepared as follows. 2.5 g of hydroxyapatite powder (Biogel-HTP, manufactured by BioRad) was put in a 50 mL tube, 20 mL of protein solution was added thereto. The resultant was left for 15 minutes, then centrifuged (2500×G, 2 minutes), and the supernatant was removed. Next, 20 mL of a stain solution was added to the precipitated hydroxyapatite. The resultant was left for 15 minutes, then centrifuged (2500×G, 2 minutes), and the supernatant was removed. After the last centrifugation, 20 mL of an artificial saliva was added to the precipitated hydroxyapatite. The resultant was left for 15 minutes, and then centrifuged (2500×G, 2 minutes). A series of operation of the above-described immersion into a protein solution, a stain solution, and an artificial saliva was repeated three times in total, the colored hydroxyapatite was suspended in a purified water, and collected on the filter paper by suction filtration, and then the collected resultant was dried at 40° C.

### Test Example 3

### Whitening Effect Evaluation Test in the Case of Addition of Ultraphosphate

20 mg of tea-stained dry hydroxyapatite powder was weighed, and H₂O₂ was added into a purified water so that the final concentration can be 3.5%, and as needed, 5% by weight of sodium ultraphosphate (manufactured by Mitejima Chemical Co., Ltd.) and 0.2% by weight of a nanocolloidal platinum catalyst were added. To the tea-stained dry hydroxyapatite, 0.3 mL of a solution that had been adjusted by the addition of a sodium phosphate buffer solution so that the pH can be 7.0 was further added and mixed well, and then the resultant mixture was incubated at 37° C. for 10 minutes. After incubation, the hydroxyapatite was precipitated by centrifugation (3000×G, 15 seconds). The supernatant was removed, and then 1 mL of a purified water was added and mixed well, and the hydroxyapatite was washed. The washing procedure with a purified water was repeated four times in total. The washed hydroxyapatite was suspended in 200 μL of a purified water, and the suspension was poured into one well of a 96-well microtiter plate. The 96-well microtiter plate was scanned from the bottom by a scanner (Epson GT8300), the obtained image was inverted to be negative, and the coloring removal rate in each treatment group was calculated from the brightness measured by image (free software).

In Table 3, the results regarding the improvement of the whitening effect of hydrogen peroxide by the addition of each kind of phosphate polymer were summarized. The values in Table 3 show the percentage of removal of the staining of stain solution from the tea-stained dry apatite in each integrant concentration.

TABLE 3

| | Nanocolloidal | Sodium | Coloring |
|---|---|---|---|
| Hydrogen peroxide | platinum | ultraphosphate | removal |
| concentration (%) | concentration (%) | concentration (%) | rate (%) |
| 0 | 0 | 0 | 0.0 |
| 3.5 | 0 | 0 | 11.0 |
| 3.5 | 0 | 5 | 54.0 |
| 3.5 | 0.2 | 0 | 11.5 |
| 3.5 | 0.2 | 5 | 62.0 |

Table 3 shows results of the comparison of whitening effect improving abilities in the cases of the addition of sodium ultraphosphate and a nanocolloidal platinum catalyst to 3.5% hydrogen peroxide. As shown in Table 3, in the case of the addition of only 0.2% nanocolloidal platinum catalyst, the coloring removal rate was 11.5%. When 5% sodium ultraphosphate was added to the solution, the coloring removal rate was 62.0%, as a result, the coloring removal rate was improved around 5.4 times as compared with that in the case without the addition of sodium ultraphosphate. As described above, it was found that even in a solution in which the concentration of hydrogen peroxide is as low as 3.5%, with the combination of a nanocolloidal platinum catalyst and sodium ultraphosphate, sufficient coloring removal effect can be obtained.

### Test Example 4

### Relationship Between the Concentration of Ultraphosphate and the Improvement of Whitening Effect

With the use of sodium ultraphosphate at various concentrations, the whitening effects of hydroxyapatite by 3.5% hydrogen peroxide and 0.2% nanocolloidal platinum catalyst were compared. At this time, treatment conditions are as follows: the pH is 7.0, the temperature is 37° C., the treatment time is 10 minutes, and the final concentration of the sodium ultraphosphate to be added was changed to 0%, 1%, 5%, and 10%, the whitening treatment was performed under these treatment conditions. After that, in the same way as in Test Example 3, the washing operation was performed, and then the coloring removal rate was calculated.

TABLE 4

| | Nanocolloidal | Sodium | Coloring |
|---|---|---|---|
| Hydrogen peroxide | platinum | ultraphosphate | removal |
| concentration (%) | concentration (%) | concentration (%) | rate (%) |
| 0 | 0 | 0 | 0 |
| 3.5 | 0 | 0 | 7.4 |
| 3.5 | 0 | 1 | 53.8 |
| 3.5 | 0 | 5 | 61.6 |
| 3.5 | 0 | 10 | 64.1 |
| 3.5 | 0.2 | 0 | 8.0 |
| 3.5 | 0.2 | 1 | 56.8 |
| 3.5 | 0.2 | 5 | 70.2 |
| 3.5 | 0.2 | 10 | 72.6 |

Table 4 shows results of the comparison of the concentration dependence of sodium ultraphosphate to the whitening effect. As a result, it was found that the higher whitening improvement effect depends on the concentration of sodium ultraphosphate. With the combination of nanocolloidal platinum catalyst and sodium ultraphosphate at a concentration of 5% or more, all the coloring removal rates exceeded 70%, and the improvement effect was 8.7 times to 9M times higher than the case of nanocolloidal platinum catalyst alone. Therefore, it was found that with the combination of nanocolloidal platinum catalyst and sodium ultraphosphate, the whitening effect can further be enhanced.

### Test Example 5

### Effect of Infrared Light on Whitening Effect

The oral composition in the above-described Examples was applied to the teeth, and then the applied area was irradiated with a laser diode for 30 seconds to 5 minutes. As the laser diode, Ezlase (registered trademark) manufactured by Waterlase Japan was used. The output wavelength of this laser diode was 940 nm, and the output intensity was 7 W. As a result, only about one minute irradiation by the laser diode can dramatically enhance whitening effect of the teeth. It would appear that whitening effect by peroxide is dramatically enhanced by the energy of the laser diode.

1. A tooth whitening composition, comprising:
0.01% by weight or more to 10% by weight or less of colloidal platinum nanoparticles, and 3.5% by weight or more to 30% by weight or less of peroxide.

2. The tooth whitening composition according to claim 1, further comprising:
1% by weight or more to 30% by weight or less of phosphate polymer, a salt of phosphate polymer, or a solvate of phosphate polymer.

3. The tooth whitening composition according to claim 2, wherein
the phosphate polymer, the salt of phosphate polymer, or the solvate of phosphate polymer is ultraphosphate, a salt of ultraphosphate, or a solvate of ultraphosphate, respectively.

4. The tooth whitening composition according to claim 1, wherein
the peroxide is hydrogen peroxide, carbamide peroxide, or calcium peroxide.

5. The tooth whitening composition according to claim 1, wherein
pH of the tooth whitening composition is 6.5 or more to 8 or less.

6. The tooth whitening composition according to claim 1, being used
by application of the tooth whitening composition on the teeth surface when used, and then
by irradiation of an applied area of the tooth whitening composition with light having a wavelength of 420 nm or more to 750 nm or less.

7. The tooth whitening composition according to claim 1, being used
by application of the tooth whitening composition on the teeth surface when used, and then
by irradiation of an applied area of the tooth whitening composition with first light having a wavelength of 420 nm or more to 490 nm or less and second light having a wavelength of 620 nm or more to 640 nm or less.

8. The tooth whitening composition according to claim 1, being used
by application of the tooth whitening composition on the teeth surface when used, and then
by irradiation of an applied area of the tooth whitening composition with light having a wavelength of 800 nm or more to 1500 nm or less.

9. A method for tooth whitening using a tooth whitening composition according to claim 1, comprising:
applying the tooth whitening composition on a teeth surface; and
irradiating an applied area of the tooth whitening composition with light.

10. The method for tooth whitening according to claim 9, wherein the irradiating is performed with a light having a wavelength in a range from 420 nm to 750 nm.

11. The method for tooth whitening according to claim 9, wherein the irradiating is performed with a first light having a wavelength in a range from 420 nm to 490 nm and a second light having a wavelength in a range from 620 nm to 640 nm.

12. The method for tooth whitening according to claim 9, wherein the irradiating is performed with a light having a wavelength in a range from 800 nm to 1500 nm.
